(19) United States
(12) Patent Application Publication
(10) Pub. No.: US 2014/0343539 A1
(43) Pub. Date: Nov. 20, 2014

(54) APPARATUSES FOR PROVIDING PROXIMAL GUIDE WIRE CONTROL, SYSTEMS INCLUDING THE SAME AND METHODS FOR INSTALLING ELONGATED MEDICAL DEVICES ONTO GUIDE WIRES

(71) Applicant: Control Medical Technology, LLC, Park City, UT (US)

(72) Inventors: Erik Scott Liddiard, West Jordan, UT (US); Shawn Fojtik, Park City, UT (US)

(73) Assignee: Control Medical Technology, LLC, Park City, UT (US)

(21) Appl. No.: 14/279,233

(22) Filed: May 15, 2014

Related U.S. Application Data
(60) Provisional application No. 61/823,675, filed on May 15, 2013.

(51) Int. Cl. A61M 25/09

(52) U.S. Cl. CPC A61M 25/0905
USPC 604/528

(57) ABSTRACT

A proximal control apparatus for providing a healthcare professional with proximal control over a guide wire and an elongated medical device, such as a catheter, installed over the catheter is disclosed. The proximal control apparatus includes a relatively short guide wire extender and a housing with a passageway for receiving the guide wire extender and a portion of an elongated medical device installed over the guide wire extender. The guide wire extender has a curvilinear configuration that corresponds to a curvilinear configuration of the passageway through the housing. These complementary configurations secure the guide wire extender within the passageway as an elongated medical instrument is installed onto and moved along the guide wire extender and a guide wire that has been coupled to a distal end of the guide wire extender.

### CROSS-REFERENCE TO RELATED APPLICATION

A claim is hereby made, pursuant to 35 U.S.C. §119(e), for priority to and the benefit of the May 15, 2013 filing date of U.S. Provisional Patent Application No. 61/823,675, titled “APPARATUSES FOR PROVIDING PROXIMAL GUIDE WIRE CONTROL, SYSTEMS INCLUDING THE SAME AND METHODS FOR INSTALLING ELONGATED MEDICAL DEVICES ONTO GUIDE WIRES,” the entire disclosure of which is hereby incorporated herein.

### TECHNICAL FIELD

This disclosure relates generally to so-called “guide wire extenders” and, more specifically, to apparatuses for effectively extending the lengths of guide wires. Further, this disclosure relates to apparatuses that provide secure proximal guide wire control while installing catheters or other elongated medical devices onto guide wires or catheters, and that include guide wire extenders that are shorter than the catheters or other elongated medical devices installed thereover.

### RELATED ART

A variety of medical procedures involve the use of guide wires, including procedures in which a catheter and/or another elongated medical instrument is introduced into the vasculature of the body of a subject. Conventionally, the length of a guide wire, from its distal end to its proximal end, is sufficient to enable the guide wire to extend from a target location within the body of a subject, through the subject's body to a location outside of the subject's body, with a proximal portion of the guide wire residing outside of the subject's body. The proximal portion of a conventional guide wire may have a length that exceeds the length of the catheter or other elongated medical instrument that is to be installed onto the guide wire and introduced into the subject's body. While the length of the proximal portion of the guide wire enables a healthcare professional to maintain control over the location of the distal end of the guide wire within the subject's body while installing the catheter or other elongated medical device onto the guide wire and while introducing the catheter or other elongated medical instrument into a subject's body, the excess length of the proximal end of the guide wire and its flexibility render it increasingly cumbersome the further the catheter or other elongated medical instrument is introduced into the subject's body. The proximal ends of guide wires often have to be gathered by an additional healthcare provider (e.g., a nurse, and assistant, etc.) as ever more distal locations along the length of the guide wire emerge from the proximal end of the catheter or other elongated medical device. Even then, it is not uncommon for the proximal ends of guide wires to fall off of tables, to be dropped on the floor or otherwise subjected to undesirable contamination that, in some cases, may necessitate replacement of the guide wire, which complicates the procedure and increases its duration and cost, as well as the risk of infection or injury to the subject during the procedure.

One solution to the problem of overly long guide wires has been the development of so-called “rapid exchange,” or “RX,” devices. Rapid exchange devices typically occupy a very small portion of the length of a guide wire and, thus, enable a health care professional to maintain a desired level of control over the guide wire while installing the rapid exchange device and introducing it into a subject's body. This feature has, in turn, resulted in shorter guide wires that are easier for a single healthcare professional to manipulate, less likely to be dropped and, thus, less likely to be contaminated or to require replacement during a procedure.

Unfortunately, shorter guide wires are not suitable for catheters or other elongated medical instruments that lack rapid exchange features—even though catheters or other elongated medical instruments that lack rapid exchange features are frequently used in conjunction with rapid exchange devices. As an example, during coronary procedures, cardiologists typically use guide wires that are 180 cm long to 200 cm long, with approximately 110 cm to approximately 140 cm of the guide wire being located within the subject's body (i.e., in vivo), while the remaining portion of the guide wire—approximately 40 cm to approximately 90 cm—resides outside of the subject's body (i.e., ex vivo). To provide a cardiologist with adequate proximal control over the guide wire as a catheter or another elongated medical instrument is installed onto the guide wire and introduced into the subject's body, the length of the proximal portion of the guide wire residing outside of the subject's body should exceed the length of a catheter or other elongated medical instrument that will reside on the guide wire, for example, by about 10 cm to about 15 cm. Thus, if, after using a guide wire for its initial intended purpose—an initial procedure, a cardiologist decides to use that guide wire to introduce a 135 cm long catheter, into the subject's body, approximately 150 cm of the guide wire should reside outside of the subject's body. However, when a 200 cm guide wire is used in the initial procedure, it may be about 55 cm to 90 cm too short to enable the cardiologist to obtain adequate proximal control while installing the 135 cm catheter for a subsequent procedure. When a 180 cm guide wire is used, it may be about 75 cm to 110 cm too short to enable the cardiologist to maintain sufficient proximal control while installing the 135 cm catheter.

To address this type of issue and similar issues, various guide wire extenders have been developed. Typically, a guide wire extender is configured to be installed on and secured to the proximal end of a guide wire that has already been introduced into a subject's body. Currently available guide wire extenders typically extend the effective lengths of short guide wires to lengths that are comparable to those of conventional long guide wires. Some guide wire extenders (e.g., guide wire extenders with lengths of 140 cm, 160 cm, etc.) may almost double the lengths of the guide wires with which they are used. While a contaminated guide wire extender may simply be disassembled from a guide wire and replaced without requiring that the entire guide wire be removed from the subject's body and that another guide wire be introduced into the subject's body, guide wire extenders create similar handling issues to those that may arise when conventional long guide wires are used (e.g., they are prone to falling off of tables, falling onto the floor, contamination, etc.; they often require handling assistance; etc.).

### SUMMARY

A proximal guide wire control apparatus, or, more simply, a “proximal control apparatus,” enables a healthcare provider to maintain proximal control over a relatively short guide wire while the healthcare provider installs an elongated medical instrument (e.g., a catheter; a rapid exchange, or “RX,” device, such as a catheter; etc.) onto the guide wire and introduces the elongated medical instrument into the body of a subject. As used herein, the terms “relatively short guide wire” and “short guide wire” encompass guide wires with lengths that will not reside within the body of a subject as intended while accommodating a catheter or other elongated medical instrument entirely outside of the body and that will not provide proximal control over the guide wire while a catheter or another elongated medical device is installed on a proximal end of the guide wire. In some embodiments, a “relatively short guide wire” or a “short guide wire” may have a length of less than 300 cm (e.g., 180 cm, 200 cm, etc.).

In various embodiments, a proximal guide wire control apparatus may include an extender and a housing for the extender.

The extender is an elongated element that is configured to be coupled to a guide wire and to enable the installation of catheters or other elongated medical devices onto the guide wire, as well as their introduction into the body of a subject. A distal end of the extender may be configured to receive and couple to a proximal end of the guide wire. In some embodiments, an outer dimension (e.g., an outer diameter, or OD, etc.) of the extender may exceed the corresponding outer dimension (e.g., outer diameter, or OD, etc.) of the guide wire. In a specific embodiment, a distal end of the extender may be configured to prevent a proximal end of a guide wire from being inserted into the distal end of the extender while a distal portion of the guide wire is straight or substantially straight, but when a distal portion of the extender is bent to a curve, the distal end of the extender may enable insertion of a proximal end of a guide wire into the distal end of the extender. The distal portion of the extender may then engage a proximal portion of the guide wire that has been introduced therein as the distal portion reassumes a straight or substantially straight configuration.

A proximal end of the extender may be configured to be received by a lumen or equivalent feature of the catheter or other elongated medical instrument. Thus, the outer dimension of the extender may be small enough to be received by the lumen or equivalent feature (i.e., it is smaller than the corresponding inner dimension (e.g., inner diameter, or ID, etc.) of the lumen or equivalent feature) of the catheter or other elongated medical instrument and to enable the catheter or other elongated medical instrument to be pushed and/or pulled over the extender. While the extender is an elongated element, it may be relatively short when compared with conventional guide wire extenders. In various embodiments, a length of the extender may be less than a length of a guide wire receptacle (e.g., a lumen, etc.) of an elongated medical instrument that will receive the extender. In some embodiments, the extender may have a length of about five centimeters (5 cm) to about twenty-five centimeters (25 cm).

The extender may be configured in a manner that enables it to be captured within the housing of the proximal guide wire control apparatus in a way that substantially prevents movement of the extender relative to the housing. In some embodiments, an extender may have a shape that includes one or more curves or bends (i.e., a non-linear shape). The bends may enable the extender to lie substantially flat, or substantially within a single plane (accounting for its outer diameter, which imparts the extender with three-dimensionality). Alternatively, the bends may impart the extender with more of a three-dimensional configuration. Additionally, the extender may be configured to substantially retain its shape, even when tensile and/or compressive forces are applied along its length and/or a flexible element (e.g., a catheter, another elongated medical instrument, etc.) is installed over it (e.g., it may be formed from a rigid material, semi-rigid material, etc.); i.e., under forces that are typically used to install a catheter or other elongated medical instrument onto a guide wire and introduce the elongated medical instrument into a subject's body.

The housing of the proximal guide wire control apparatus may include a passageway that is configured complementarily to the extender and to receive at least a portion of the length of the extender. An inner dimension (e.g., an inner diameter, or ID, etc.) of the passageway may be larger than the corresponding outer diameter (e.g., outer diameter, or OD, etc.) of the extender, which may provide for a gap or space between the surface of the passageway, or the inner surface of the housing, and the outer surface of the extender. In some embodiments, that gap, as well as the relative dimensions of the passageway and extender, may be sufficient to accommodate a catheter or other elongated medical instrument, and enable the elongated medical instrument to simultaneously slide over the extender and through the passageway of the housing.

The configuration of the passageway of (or through) the housing may also enable the housing to capture the extender in a manner that limits or substantially prevents the extender from moving relative to the housing (e.g., with differences between an inner dimension (e.g., inner diameter, or ID, etc.) of the passageway and an outer dimension (e.g., outer diameter, or OD, etc.) of the extender enabling some minimal movement of the extender relative to the housing, etc.). Together, the shape and relative rigidity of the extender and the configuration of the passageway through the housing may substantially prevent movement of the extender relative to the housing when the extender and housing are assembled with one another.

In some embodiments, the proximal end of the extender may extend substantially to or completely to a corresponding end or edge of the housing. In other embodiments, the proximal end of the extender may be configured to protrude from a corresponding end or edge of the housing. These configurations may facilitate installation of a distal end of a catheter or other elongated medical instrument onto the proximal end of the extender, and introduction of the distal end of the elongated medical instrument into the passageway of the housing.

The location of the distal end of the extender relative to its corresponding end or edge of the housing may enable coupling of the extender to the proximal end of a guide wire while the extender resides within the passageway of the housing. In some embodiments, where manual manipulation of the distal end of the extender is not necessary to couple the extender to a guide wire, the distal end of the extender may be located within the passageway of the housing, at or near a corresponding end or edge of the housing. Alternatively, the distal end of the extender may protrude from its corresponding end or edge of the housing, which may enable manual manipulation of the distal end of the extender (e.g., ending, holding, squeezing, etc.) to facilitate coupling of the distal end to the proximal end of a guide wire.

The housing may be configured in a way that enables a user, such as a healthcare professional, to easily and readily access the entire length of the passageway of (or through) the housing and any extender within the passageway. Such a housing may enable a user to select an extender having desired dimensions (e.g., a distal end with an inner diameter that will receive and couple to a guide wire of a particular type or with a particular outer diameter at its proximal end; an outer diameter that enables the extender to fit within the lumen of a selected catheter or other elongated medical instrument; etc.) and to readily introduce the selected extender into the passageway. Alternatively, the housing may comprise a disposable element that is pre-loaded with an extender, and that has a configuration that does not facilitate ready removal of the extender from the passageway or ready replacement of another extender in the passageway.

The housing may be configured to be held by an individual's hand and, thus, to enable the individual to hold the extender in place as the extender and housing are used to install a catheter or other elongated medical device onto the extender or onto a guide wire to which the extender is secured, and while the elongated medical instrument is introduced into the body of a subject. Optionally, the housing may be configured to rest on a table or other flat surface in a manner that supports the housing and any other apparatuses that have been assembled with the housing (e.g., the extender, a guide wire, a catheter or other elongated medical instrument, etc.) and, thus, in a manner that prevents dropping of the housing and any other apparatuses assembled therewith, as well as contamination that may occur of the housing and any other apparatuses assembled therewith are dropped.

Various embodiments of a system for introducing an elongated medical instrument, such as a catheter, into the body of a subject include an embodiment of an extender that incorporates one or more teachings of this disclosure, an embodiment of a housing that incorporates one or more teachings of this disclosure and one or both of another extender, a guide wire and an elongated medical instrument.

In another aspect, a method for maintaining proximal control over a relatively short guide wire while installing an elongated medical instrument, such as a catheter, on the guide wire and/or introducing the elongated medical instrument into the body of a subject is disclosed. Such a method includes securing a distal end of a short, non-linear extender to a proximal end of a guide wire. A majority of the length of the guide wire may reside within the subject's body when the extender is secured to the guide wire.

The extender is disposed within a complementary passageway of a housing in a manner that substantially prevents movement of the extender relative to the housing. In addition, the extender may substantially retain its shape under the forces that are typically needed to install an elongated medical instrument on a guide wire and introduce the elongated medical instrument into the body of a subject, further securing the extender within the passageway of the housing. In some embodiments, an individual (e.g., a health care provider, an assistant, etc.) may select an extender (e.g., an extender having a desired outer dimension, an extender configured to be coupled to a guide wire of a particular type or with a particular outer dimension, etc.) and introduce the extender into the passageway of the housing in a manner that substantially prevents movement of the extender relative to the housing.

With the extender in the housing, an individual (e.g., a health care professional, an assistant, etc.) may hold the housing and, while holding the housing, grasp an elongated medical instrument to assemble the same with the extender. More specifically, a proximal end of the extender may be introduced into a distal end of a lumen or other receptacle of the elongated medical instrument, or the elongated medical instrument may be installed on the proximal end of the extender. In addition, as an individual holds the housing, the distal end of the elongated medical instrument may be introduced into and through the passageway of the housing, out of a second location of the housing (e.g., an opposite side of the housing, etc.) and onto the guide wire, then introduced into the subject's body.

When a guide wire is positioned within a subject's body to enable the performance of an initial procedure, the guide wire may remain in place during the initial procedure and a health care professional decides to perform an additional procedure at the same site within the subject's body, the guide wire may remain in place within the subject's body following the initial procedure and used to enable performance of the additional procedure. More specifically, a proximal guide wire control apparatus may be used in a manner that enables the guide wire to be used in two or more procedures, regardless of the lengths of the elongated medical devices that are to be introduced into the subject's body along the guide wire to effect the different procedures.

Other aspects, as well as features and advantages of various aspects, of the disclosed subject matter will become apparent to those of ordinary skill in the art through consideration of the ensuing description, the accompanying drawings and the appended claims.

### BRIEF DESCRIPTION OF THE DRAWINGS

In the drawings:

FIG. 1 illustrates an embodiment of a proximal control apparatus that includes an extender for a guide wire and a housing that defines a passageway that is configured complementarily to the extender, with the housing in an open orientation that enables placement of the extender within the passageway and removal of the extender from the passageway;

FIG. 2 depicts the embodiment of proximal control apparatus shown in FIG. 1, with a distal end of the extender coupled to a proximal end of a guide wire and the housing in a closed orientation, which traps the extender within the passageway of the housing;

FIGS. 2A through 2C are cross sectional representations of an embodiment of a distal portion of an extender that depict an embodiment of a guide wire engagement element, as well as an embodiment of the manner in which a proximal portion of a guide wire may be introduced into the distal portion of the extender and engaged by the guide wire engagement element;

FIG. 3 shows installation of an embodiment of an elongated medical instrument (in this case, a catheter) onto a proximal end of the extender shown in FIGS. 1 and 2 through 2C;

FIG. 4 depicts continued distal movement of the distal end of the elongated medical instrument of FIG. 3 along the extender, through the passageway of the housing of the proximal control apparatus shown in FIGS. 1, 2 and 3 and over a proximal end of a guide wire;

FIG. 5 shows a proximal end of the extender emerging from a proximal end of the elongated medical instrument; and

FIG. 6 shows the housing in an open orientation, which enables removal of a proximal end of the elongated medical instrument from the housing; the extender and the guide wire may be moved relative to the elongated medical instrument or removed from the elongated medical instrument to enable a healthcare professional to use the elongated medical instrument to perform one or more desired functions.

### DETAILED DESCRIPTION

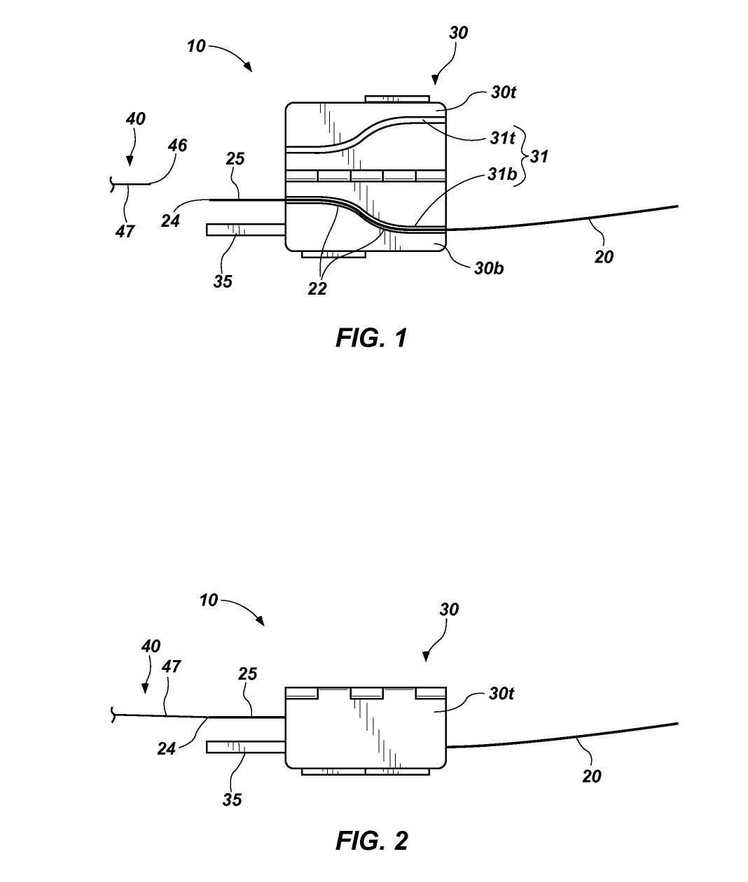

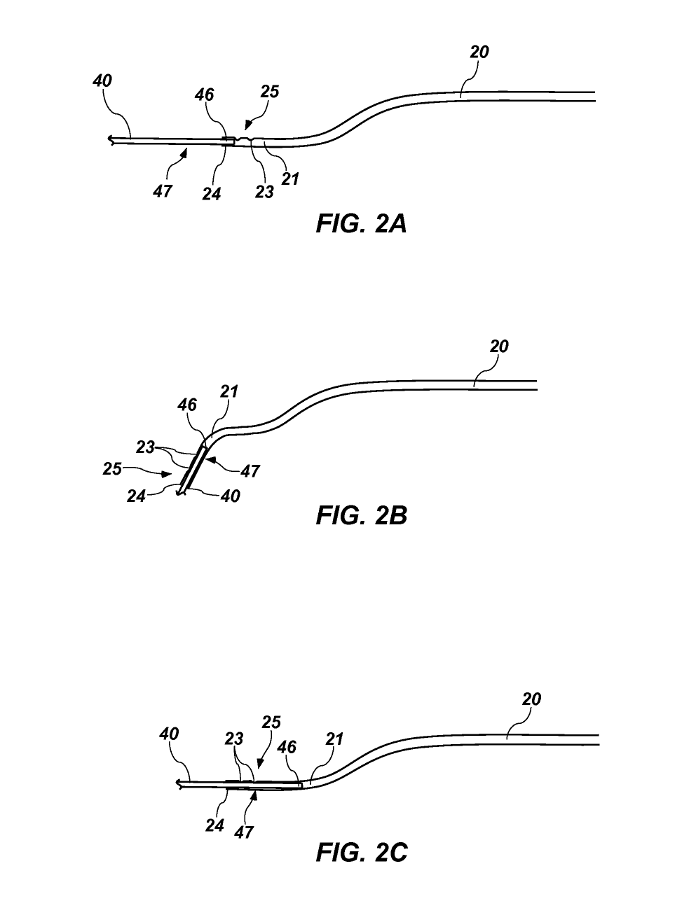

FIGS. 1 and 2 illustrate an embodiment of a proximal control apparatus 10. The proximal control apparatus 10 includes an extender 20 and a housing 30. In addition to the proximal control apparatus 10, FIGS. 1 and 2 show an embodiment of a guide wire 40 with which the proximal control apparatus 10 may be used.

The extender 20 of the embodiment of proximal control apparatus 10 shown in FIGS. 1 and 2 is elongated, but relatively short. In some embodiments, the extender 20 may have a length of about 5 cm to about 25 cm. In addition, the extender 20 has a non-linear shape and, thus, includes at least one curve or bend 22. Each curve or bend 22, as well as the arrangement of two or more curves or bends 22 along the length of the extender 20, may enable an elongated medical instrument (not shown in FIG. 1 or FIG. 2), such as a catheter, to move over the extender 20 in a substantially continuous manner when a constant amount of longitudinal force is applied to the elongated medical instrument and/or to the extender 20.

The extender 20 may be configured to substantially retain its shape, even when tensile and/or compressive forces are applied along its length and/or a flexible element (e.g., a catheter, another elongated medical instrument, etc.) is installed over it and/or forced (distally or proximally) thereover; i.e., under forces that are typically used to install a catheter or other elongated medical instrument onto a guide wire and introduce the elongated medical instrument into a subject's body. In some embodiments, the extender 20 may be semi-rigid, substantially rigid or rigid.

The extender 20 may be tubular in shape or have a similar shape. As an alternative, a distal portion 25 of the extender 20 may be tubular, while one or more other portions of the extender 20 (e.g., a remainder of the extender, etc.) are solid or substantially solid.

A distal end 24 and/or a distal portion 25 of the extender 20 may be configured to engage or otherwise couple with a proximal end 46 or a proximal portion 47 of a guide wire 40. In this regard, the distal end 24 and/or distal portion 25 of the extender 20 may include one or more coupling features that are configured to mechanically couple with the proximal end 46 and/or the proximal portion 47 of the guide wire 40. Various configurations of coupling features include, but are not limited to the following: features that establish an interference fit between the distal end 24 and/or distal portion 25 of the extender 20 and the proximal end 46 and/or a proximal portion 47 of the guide wire 40; interlocking features, e.g., features of the distal end 24 and/or distal portion 25 of the extender 20 that are configured to interlock with features of the proximal end 46 and/or a proximal portion 47 of the guide wire 40, or so-called slot-key mechanisms; and complementarily threaded elements.

An embodiment of coupling features of the distal portion 25 of the extender 20 that may engage the proximal portion 47 of a guide wire 40 by an interference fit is shown by FIG. 2, and by FIGS. 2A through 2C. The extender 20, or at least a distal portion 25 thereof, may have a tubular or similar configuration with a conduit 21 that extends through the distal portion 25 and opens to the distal end 24. Thus, the extender 20, or at least its distal portion 25, may be configured to receive a proximal end 46 and proximal portion 47 of a guide wire 40. One or more guide wire engagement elements 23, which may comprise indented locations of a wall of the extender 20, may protrude into the conduit 21. In the illustrated embodiment, the extender 20 includes two guide wire engagement elements 23 that are aligned with one another along a generatrix of (e.g., a line along a length of, etc.) the distal portion 25 of the extender 20. As shown in FIG. 2A, when the distal portion 25 of the extender 20 is in a substantially straight configuration or a straight configuration (which may be its configuration when no transverse force is applied to the distal portion 25), each guide wire engagement element 23 may protrude a sufficient distance into the conduit 21 to prevent the proximal end 46 and proximal portion 47 of the guide wire 40 from being introduced into the conduit 21. When a transverse force is applied to the distal portion 25 of the extender 20 in a way that causes the distal portion 25 to assume a curved configuration, or flex, with each guide wire engagement element 23 on an outside of the curve, as illustrated by FIG. 2B (e.g., to a guide 35 on the housing 30 shown in FIG. 2, etc.), the force of the curve on each guide wire engagement element 23 stretches each guide wire engagement element 23, reducing the distance it protrudes into the conduit 21 and opening the conduit 21 enough to enable insertion of the proximal end 46 and the proximal portion 47 of the guide wire into the conduit 21. When the transverse force is released, the distal portion 25 of the extender 20 may re-assume its substantially linear or linear configuration, enabling the distance each guide wire engagement element 23 protrudes into the conduit 21 to increase and, thus, to engage an adjacent portion of the proximal portion 47 of the guide wire 40 in a manner that will securely hold the proximal portion 47 of the guide wire 40 within the conduit 21.

An outer dimension of the extender 20 (e.g., its outer diameter, or OD, etc.) is configured to enable the extender to be received by a lumen or other receptacle of an elongated medical instrument, such as a catheter.

With returned reference to FIG. 1, the housing 30 includes a passageway 31 that is configured complementarily to, and to receive, a corresponding extender 20. Thus, the passageway 31 is configured to receive a corresponding, complementary extender 20. In addition, the passageway 31 may have dimensions (e.g., a diameter, etc.) sufficient to enable an elongated medical instrument (not shown in FIG. 1 or FIG. 2) installed over an extender 20 to reside within the passageway 31 and to be forced through the length of the passageway 31. Thus, an inner dimension of the passageway 31 may be slightly larger than an outer dimension of an elongated medical instrument with which the housing 30 and extender 20 are configured to be used.

Additionally, the complementary configurations of the passageway 31 and a complementary extender 20 may enable the passageway 31 to substantially prevent movement of an extender 20 disposed therein.

In the depicted embodiment, the housing 30 includes two halves 30t and 30b that may be assembled with one another. Each half 30t, 30b defines a portion of the passageway 31t, 31b, respectively. When the halves 30t and 30b of the housing 30 are placed in an assembled relationship, the portions 31t and 31b align with one another to define the passageway 31. In the specific embodiment depicted by FIGS. 1 and 2, the halves 30t and 30b of the housing 30 are hingedly connected to each other (e.g., by way of a pivoting hinge, a living hinge, etc.) to enable the housing to open and close to expose the extender 20 and any wire connected to it in a manner that ensures that they are properly assembled and that the portions 31t and 31b of the passageway 31 are properly aligned when the halves 30t and 30b are positioned in the assembled relationship. Of course, a housing 30 may include other features for ensuring proper alignment of the halves 30t and 30b.

In embodiments where the housing 30 of a proximal control apparatus 10 includes two halves 30t and 30b or a similar configuration, an extender 20 may be introduced into an exposed portion of a passageway 31 (e.g., in portion 31t, in portion 31b, etc.), as depicted by FIG. 1. The halves 30t and 30b of the housing 30 may then be placed and secured in the assembled relationship, as illustrated by FIG. 2, effectively enclosing the extender 20 (or a portion thereof) within the passageway 31 and, thus, holding the extender 20 in place relative to the housing 30.

The housing 30 may be configured in a manner that enables it to be stably supported by a flat surface, such as a tabletop or the surface of a cart. Such a configuration may enable the housing 30 to rest upon and/or to be held against a flat surface during use of the housing 30 and an extender 20 that has been assembled with the housing 30. Optionally, such a configuration may prevent the housing 30 from inadvertently coming off of the flat surface (e.g., by rolling or otherwise). In the depicted embodiment the housing 30 is (and its halves 30t and 30b are) substantially flat.

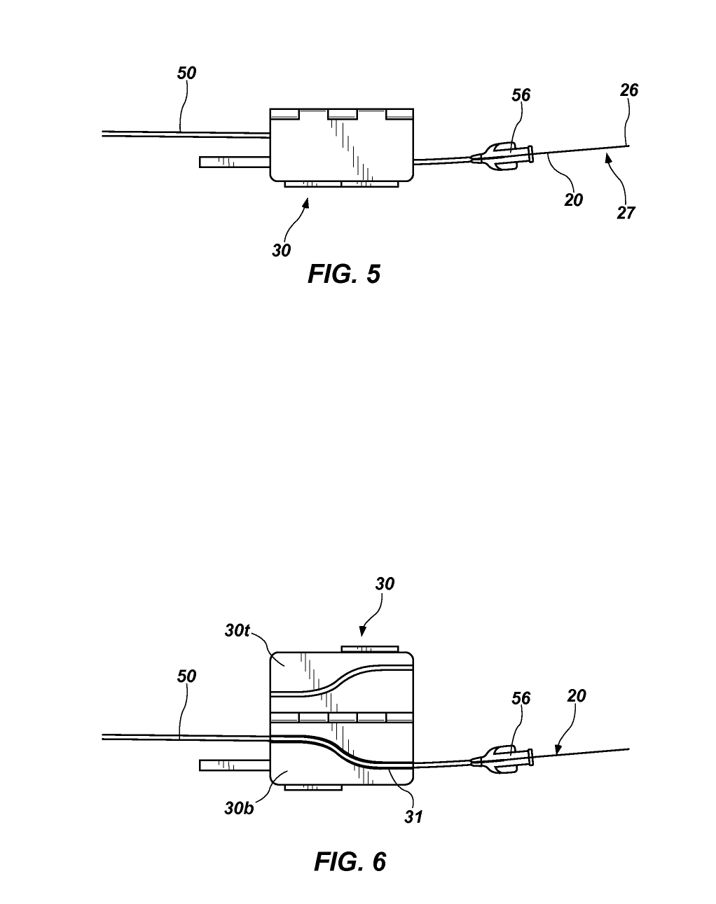

In the illustrated embodiment, the extender 20 is longer than the passageway 31 of, or through, the housing 30, with each end 24 and 26 (FIG. 5) of the extender 20 protruding beyond its corresponding edge or end of the housing 30. Alternatively, one or both ends 24, 26 of an extender 20 may only extend to or substantially to a corresponding edge or end of the housing 30. In any event, the relative position of at least the proximal end 26 of the extender 20 relative to its corresponding edge or end of the housing 30 may facilitate installation of a distal end of an elongated medical instrument (not shown in FIG. 1 or FIG. 2) onto the proximal end 26 of the extender 20, as well as the introduction of the distal end of the elongated medical instrument into the passageway 31 of the housing 30.

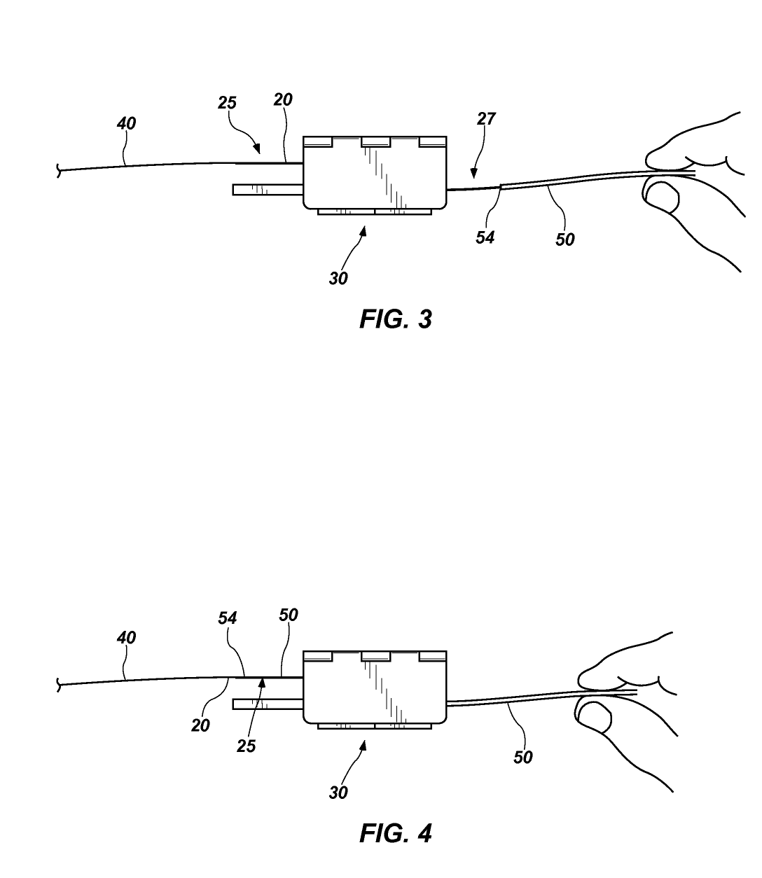

With continued reference to FIGS. 1-2C, a guide wire 40 may be introduced into the body of a subject in a suitable manner. With the guide wire 40 in place, a proximal portion 47 of the guide wire 40 may be coupled with a distal portion 25 of an extender 20, which may be at least partially disposed within and captured by a passageway 31 of a housing 30 of a proximal control apparatus 10. While the housing 30 and a configuration of the extender 20 substantially prevent movement of the extender 20 relative to the housing 30, the proximal control apparatus 10 may be used to introduce an elongated medical instrument 50 onto the guide wire 40 and into the body of the subject, as illustrated by FIGS. 3 through 6.

FIG. 3 depicts the installation of an elongated medical instrument 50 onto the extender 20. More specifically, a receptacle, such as a lumen, at a distal end 54 of the elongated medical instrument 50 is inserted onto a proximal portion 27 of the extender 20, over a proximal end 26 (FIG. 5) of the extender 20; i.e., the proximal end 26 of the extender 20 is inserted into the receptacle at the distal end 54 of the elongated medical instrument 50. As the elongated medical instrument 50 moves distally onto and over the proximal portion 27 of the extender 20, the housing 30, which may be secured in place (e.g., by an individual's hand, etc.) or, optionally, rest upon a support surface, such as a table or a cart, holds the extender 20 in place. In turn, the extender 20 holds a guide wire 40, which has been coupled to a distal portion 25 of the extender 20, in place.

With continued distal movement of the elongated medical instrument 50 along the extender 20, as illustrated by FIG. 4, the distal end 54 of the elongated medical instrument 50 is introduced into and through the passageway 31 (FIG. 1) of the housing 30. The housing 30 and its passageway 31 may continue to hold the extender 20 and, thus, the guide wire 40 that has been coupled to the distal portion 25 of the extender 20 in place as the elongated medical instrument 50 moves distally over the extender 20 and through the passageway 31 (FIG. 1) of the housing 30.

Further distal movement of the elongated medical instrument 50 causes it to move onto and over the guide wire 40 (FIG. 4), as shown in FIG. 5. As the elongated medical instrument 50 moves over the guide wire 40, it is introduced into the body of a subject. With such movement, the extender 20 and the housing 30 enable a user (e.g., a healthcare professional, an assistant, etc.) to maintain proximal control, even manual proximal control, over the guide wire 40.

Ultimately, a proximal end 56 of the elongated medical instrument 50 may move beyond the proximal end 26 and the adjacent proximal portion 27 of the extender 20. As illustrated by FIG. 6, with the elongated medical instrument 50 in place within a subject's body, the elongated medical instrument 50 and the extender 20 may be removed from the passageway 31 of the housing 30. Optionally, the extender 20 and the guide wire 40 (FIG. 4) may be removed from the elongated medical instrument 50. With the housing 30 removed (and, optionally, the extender 20 and the guide wire 40 also removed), a healthcare professional may use the elongated medical instrument 50 to perform an appropriate medical procedure.

A proximal control apparatus 10 may enable the effective extension of a guide wire 40 and the introduction of an elongated medical instrument 50 into the body of a subject more quickly than a conventional guide wire extender may be assembled with a guide wire and used to introduce the elongated medical instrument 50 into the subject's body. In addition, use of a proximal control apparatus 10 may reduce the likelihood of contamination (e.g., through dropping the guide wire, guide wire extender and/or elongated medical instrument onto the floor or another contaminated surface, etc.) that accompanies the use of conventional, long guide wires and conventional guide wire extenders to introduce elongated medical instruments into the body of a subject. Furthermore, by eliminating most of the length of a conventional guide wire extender, a proximal control apparatus 10 (including its extender 20 and housing 30) may also be less expensive to manufacture than conventional guide wire extenders.

Although the foregoing description sets forth many specifics, these should not be construed as limiting the scope of any of the claims, but merely as providing illustrations of some embodiments and variations of elements or features of the disclosed subject matter. Other embodiments of the disclosed subject matter may be devised which do not depart from the spirit or scope of any of the claims. Features from different embodiments may be employed in combination. Accordingly, the scope of each claim is limited only by its plain language and the legal equivalents thereto.

What is claimed:

1. A proximal control apparatus for providing proximal control over a short guide wire, the proximal control apparatus comprising:
an extender comprising an elongated element:
having a length that is less than a length of a guide wire receptacle of an elongated medical instrument with which the extender is to be used;
having a configuration, including at least one curve, that enables a position of the extender to be substantially retained;
being configured to substantially retain the configuration, including the at least one curve when the elongated medical instrument is installed on the extender; and
including a distal end or a distal portion configured to couple with a proximal end or a proximal portion of a guide wire; and
a housing defining a passageway, the passageway:
having a configuration complementary to the configuration of the extender;
configured to receive at least a portion of the extender, including the at least one curve, and to substantially retain the position of the extender; and
having an inner dimension that enables the elongated medical instrument to reside over at least a portion of the extender within the passageway while the passageway substantially retains the position of the extender.

2. The proximal control apparatus of claim 1, wherein the extender has a length of about 5 cm to about 25 cm.

3. The proximal control apparatus of claim 1, wherein the distal end or the distal portion of the extender includes a coupling feature that enables the distal end or the distal portion to couple with the proximal end or the proximal portion of the guide wire.

4. The proximal control apparatus of claim 3, wherein the coupling feature is configured to establish at least one of an interference fit, an interlocked relationship or a threaded engagement between the distal end or the distal portion of the extender and the proximal end or the proximal portion of the guide wire.

5. The proximal control apparatus of claim 3, wherein the coupling feature includes:
a receptacle within at least the distal portion of the extender, the receptacle configured to receive the proximal portion of the guide wire; and
at least one engagement element protruding into the receptacle, the at least one engagement element configured to recede and to enable insertion of the proximal portion of the guide wire into the receptacle as a force is applied to the distal portion of the extender to cause the distal portion to curve with the at least one engagement element at an outside of the curve and to protrude as force is removed from the distal portion of the extender and the distal portion substantially assumes its normal configuration.

6. The proximal control apparatus of claim 5, wherein the housing includes a guide configured for use in applying the force to the distal portion of the extender.

7. The proximal control apparatus of claim 1, wherein the housing includes a plurality of elements that are configured to be placed in an assembled relationship with one another, each of the plurality of elements defining a portion of the passageway, different portions of the passageway being placed in communication with one another upon placement of the plurality of elements of the housing in the assembled relationship.

8. The proximal control apparatus of claim 7, wherein, when the housing is in a disassembled relationship, a portion of the passageway is exposed to enable assembly of the extender with that portion of the passageway or disassembly of the extender from that portion of the passageway.

9. A method for introducing an elongated medical instrument into a body of a subject, comprising:
introducing a guide wire into a body of a subject, a distal end of the guide wire located at or proximate to a target location within the body, a proximal portion of the guide wire residing outside of the body and having a length that is about the same as or less than a length of a guide wire receptacle of an elongated medical instrument to be introduced into the body over the guide wire;
coupling an extender having a length that is less than a length of the guide wire receptacle of the elongated medical instrument with a proximal end or a proximal portion of the guide wire, the extender having a configuration that includes at least one curve and being configured to substantially retain the configuration as the elongated medical instrument is installed on the extender;
capturing the extender within a passageway of a housing for the extender, the housing configured to substantially hold the extender in place as the elongated medical instrument is installed on the extender or the guide wire or introduced into the body on the guide wire;
installing the elongated medical instrument onto a proximal portion of the extender;
while the extender is captured within the passageway of the housing, moving the elongated medical instrument distally over the extender and through the passageway, over and distally along the guide wire and into the body.

10. The method of claim 9, wherein coupling includes establishing an interference fit between a distal portion of the extender and the proximal portion of the guide wire.

11. The method of claim 10, wherein establishing an interference fit comprises:
bending the distal portion of the extender in a manner that reduces a distance at least one engagement element protrudes into a receptacle of the distal portion;
with the distal portion bent, introducing the proximal portion of the guide wire into the distal portion of the extender; and
removing a bending force from the distal portion of the extender to allow the distal portion to substantially assume a normal, unstressed configuration, increasing the distance the at least one engagement element protrudes into the recess and enabling the at least one engagement element to engage the proximal portion of the guide wire and to secure the proximal portion within the receptacle.

12. The method of claim 11, wherein bending the distal portion of the extender comprises bending the distal portion of the extender to define a curve, with the at least one engagement element on an outside of the curve.

13. The method of claim 9, wherein capturing includes:
introducing at least a portion of the extender into a complementarily configured passageway of the housing.

14. The method of claim 13, wherein introducing comprises introducing at least the portion of the extender into a portion of the passageway defined by an unassembled member of the housing.

15. The method of claim 14, wherein capturing further includes:
placing the unassembled member of the housing and another member of the housing in an assembled relationship in which the portion of the passageway communicates with another portion of the passageway, the assembled relationship of the member and the another member defining the passageway within which the extender is positioned and capturing the extender within the housing.

16. The method of claim 9, further comprising:
holding the housing while installing the elongated medical instrument onto the proximal portion of the extender and while moving the elongated medical instrument distally.

17. The method of claim 16, wherein holding the housing comprises securing the housing in a substantially stationary position.

18. The method of claim 9, further comprising:
removing the extender and a portion of the elongated medical instrument on the extender from the passageway of the housing.

19. The method of claim 18, further comprising:
removing the extender and the guide wire from the elongated medical instrument.

20. The method of claim 18, further comprising:
performing a medical procedure with the elongated medical instrument.
